(19) United States
(12) Patent Application Publication
(10) Pub. No.: US 2021/0238434 A1
(43) Pub. Date: Aug. 5, 2021

(54) DNA-TAGGED INKS AND SYSTEMS AND METHODS OF USE

(71) Applicant: VIDEOJET TECHNOLOGIES INC., Wood Dale, IL (US)

(72) Inventors: Michael KOZEE, Wheaton, IL (US); Linfang ZHU, Woodridge, IL (US); Zheng XUE, Burr Ridge, IL (US); Lawrence JUNG, Stony Brook, NY (US)

(21) Appl. No.: 16/972,760

(22) PCT Filed: Jun. 6, 2019
(86) PCT No.: PCT/US2019/035705

§ 371 Date: Dec. 7, 2020

Related U.S. Application Data
(60) Provisional application No. 62/681,999, filed on Jun. 7, 2018.

(51) Int. Cl. C09D 11/36; C09D 11/108; C09D 11/50; C12Q 1/6806

(52) U.S. Cl. CPC C09D 11/36; C12Q 1/6806; C09D 11/50; C09D 11/108

(57) ABSTRACT

The invention provides stable nucleic acid-tagged ink compositions suitable for printing an ink mark on an object as an identifier or for authentication of goods, and methods of marking an article with such a mark. These ink compositions contain a specific nucleic acid of known length and sequence that can be deposited on an article by inkjet printing or thermal transfer printing to produce a mark, which may be visible or invisible, covert or overt. These ink products are stable and can accurately deliver an amount of nucleic acid tag that can be removed or sampled and analyzed by known DNA analytical methods to detect the specific nucleic acid on the marked article but which cannot be identified without previous knowledge of the nucleic acid sequence in the mark. The invention also provides methods of applying these tagged marks using inkjet printers and printer systems, and the systems for using the tagged ink compositions.

### CROSS-REFERENCE TO RELATED APPLICATIONS

This application claims the benefit of U.S. provisional application Ser. No. 62/681,999, filed 7 Jun. 2018. The entire contents of this application is hereby incorporated by reference as if fully set forth herein.

### BACKGROUND

### 1. Field of the Invention

The present invention relates to inks, methods and systems employing nucleic acid (preferably DNA) tags and methods for using inks and systems for applying tagged marks, preferably used in inkjet printing.

### 2. Background of the Invention

Worldwide, pirated, copied and falsely-sold goods presents a problem that ranges from a serious business risk to an outright existential challenge. Public institutions like national defence and public health care, and any number of the private enterprises that support them such as pharmaceuticals, arms production, etc. are harmed by the increasing prevalence of such falsified goods. Beyond copies or fakes, companies often struggle to simply stop the sale of goods in certain regions which were intended for sale in other places (grey marketing) or to stop the sale of goods where they are not intended to be sold (black marketing).

One of the first lines of defence for protecting goods is proper labeling, however customers and rogue distributors of goods often are not troubled by overt differences in labeling and labels are easily copied or authentic packages are simply refilled with counterfeit material. In addition, one strategy is to include specialized security features that can help to enable proper identification of materials. Security features that are more difficult to copy (e.g., computer chip and encryption technologies) typically are very expensive and therefore unsuitable for anything but very high value items and are only implementable at a case/multiple packaging level. These solutions, too, still can be copied with enough effort and investment. Often because differences in local value of goods can be several fold, the potential profit from such activities warrants very aggressive copying or repackaging. The track record for existing security features to resist copying once detected, unfortunately, is very poor and thus a whole industry has arisen based on delivering multiple security features that can be adapted in response to these persistent, illicit efforts.

Less expensive solutions based on security tags that can be applied directly onto the product, incorporated within the product, or as part of the primary packaging of the product, are typically very difficult to implement because no suitable printing/delivery solution exists. In addition, these solutions are usually either not highly differentiated (e.g., not many different unique tags exist) and/or they are easily copied.

Using serialized codes along with database validation can solve some of the above challenges and can be especially cost effective. However, databases require encryption to be secure and are otherwise easily copied, in part because databases must be accessible or cloud-based. Also, data tends to be more difficult to use during litigation as proof of copying than physical identification because it is less tangible. For some customers, serialization does not go far enough, especially given mandates such as the U.S. Drug Quality Security Act and similar worldwide regulations that increasingly place the burden of identifying and reporting counterfeits within the supply chain on government agencies and within a prescribed time limit.

The inability to do a ‘second check’ of authenticity in addition to existing serialization/identification methods leaves added room for doubt whenever a question about the authenticity of items arises, especially if the tags/codes are not part of the actual item. Hence for certain customers, there remains a need to print a security mark directly on manufactured articles that can be validated unequivocally by analysis.

In addition, some articles cannot be effectively serialized at the primary product level due to process incompatibility with printing or physical marking methods, poor ink adhesion, lack of space to apply a printed mark, the concern that an identifying mark would alter product appearance or functionality, etc. For example, inkjet printing is widely used on the primary and secondary packaging of products to print variable information such as an expiration date, production batch number, bar codes, and the like.

A typical way to incorporate a security tag to help identify the authenticity of the products or to augment a serialization scheme can be to simply dose these inkjet codes with a chemical security tag. However, problems exist with conventionally available security tags. Many of these comprise heavy pigments with large particle sizes that tend to settle out, leading to printer operational issues and reduction of the amount of the security pigment in the printed codes over time. Other available tags based on organic dyes are usually not stable enough after exposure in the environment, particularly after exposure to light, heat, and oxygen in the air, etc. It is also very difficult to customize the security feature for different customers as there are limited numbers of unique pigments and dyes.

Hence there is a need for a printing method compatible with production environments to deliver a mark with good adhesion to a variety of kinds of products that contains a security tag. This is especially true for small components, products with a nonporous surface, and products made using high speed production processes, etc. Such a tag would be both stable and inkjet compatible, and easily customizable.

### SUMMARY OF THE INVENTION

Therefore, the ink compositions according to the invention are tagged with a nucleic acid of known length and sequence or for which an amplification method is known. The particular length and sequence of the nucleic acid is not critical to the invention, as long as either the length and sequence are known, or a method for its amplification is known (for example suitable primers exist). The particular nucleic acid tag preferably is associated with a particular article and serves to identify that article or to identify that article as authentic, when applied to the article or its packaging as a printed mark.

In particular, the invention provides a tagged ink composition, comprising: about 35% to about 95% of a volatile organic solvent; and an amplifiable nucleic acid tag. In some embodiments, the tagged ink composition does not contain a stabilizing agent or a solubilizing agent; in some embodiments, the tagged ink composition contains a stabilizing agent or a solubilizing agent which is a volatile organic solvent.

Preferred tagged ink compositions are formulated for inkjet printing or thermal transfer printing.

Preferred solvents for use with the tagged ink compositions include one or more of the group consisting of a ketone, an alcohol, an ester, and an ether. Most preferred solvents for the tagged ink compositions are methanol, ethanol, methyl ethyl ketone, acetone, or a mixture thereof. Preferred tagged ink compositions comprise less than 10% added water, more preferably less than 5% added water, and most preferably less than 1% added water.

In general, preferred tagged ink compositions as described above contain as the nucleic acid, double-stranded DNA, which preferably has a molecular weight less than or equal to 650 kDa, or has a molecular weight less than or equal to 65 kDa, or has a molecular weight less than or equal to 32 kDa, or has a molecular weight less than or equal to 6 kDa. Preferred DNA for use in the invention is at least 10 base pairs long, or is at least 50 base pairs long, or is at least 100 base pairs long, or is at least 1000 base pairs long.

The tagged ink compositions preferably have a nucleic acid concentration which is less than or equal to 0.1% by weight, preferably less than or equal to 1 part per million, more preferably less than or equal to 100 parts per billion, and most preferably less than or equal to 1 part per billion. In highly preferred tagged ink compositions, the nucleic acid has a molecular weight of less than 100 kDa and the nucleic acid concentration in the ink composition is less than 0.1% by weight.

The preferred tagged ink compositions are those both wherein the nucleic acid is substantially soluble or dispersed within the ink composition, and wherein the composition is stable and the nucleic acid is detectable in a printed mark after bottle storage for at least 1 year and during operation within a continuous inkjet printer for more than 900 hours.

In some embodiments, the tagged ink compositions further comprise a resin, preferably wherein the resin is a thermoplastic resin that develops good permanence and adhesion after solvent evaporation. The resin, in some embodiments, is cross-linkable or thermally curable; optionally the thermally curable resin is curable by UV or UV LED irradiation. Preferred resins include but are not limited to an acrylic resin, a vinyl chloride/vinyl acetate copolymer, a polyester, a polyvinyl butyral resin, an epoxy-phenolic resin, a functionalized silicone resin, a cellulose acetate propionate, a cellulose acetate butyrate, a polyurethane resin, a modified rosin resin, a phenolic resin, a polyamide, a ethyl cellulose, a cellulose ether, a cellulose nitrate, a polymaleic anhydride, a acetal polymer, a styrene/methacrylate copolymer, a aldehyde resin, a ketone resin, a copolymer of styrene and allyl alcohol, a polyhydroxystyrene, a polyketone, a sulfonamide-modified epoxy resin, a terpene phenolic resin, a modified cellulose, a shellac, a polyvinyl pyrrolidine, and any combination thereof. More preferred tagged ink compositions contain a styrene acrylic resin or a nitrocellulose resin.

The tagged ink compositions optionally further comprise a colorant, which can be visible or invisible to the naked eye, or the colorant is a luminescent compound which can be visible or invisible to the naked eye.

The invention also relates to a method of tagging an article, comprising applying the tagged ink compositions as described above to the article or packaging for the article to produce a mark. The tagged ink composition can be applied during manufacture of the article or the subsequent packaging step or applied after manufacture of the article or the packaging; and in some embodiments is applied directly onto the article or a component of the article.

In certain embodiments, the methods are those wherein the tagged ink composition is applied by inkjet printing or thermal transfer printing to produce a mark, preferably by a non-contact method such as inkjet. A particularly preferable inkjet method to produce a mark is continuous inkjet printing. In embodiments of the method, the tagged ink composition is applied in ink droplets ranging in size from 0.1 nL to 10 nL.

Embodiments of the methods can produce a mark that is overt, or covert, or likewise a code that is either overt or covert. Optional methods further comprise applying a solvent-resistant coating over the printed mark. The printed marks produced by the inventive methods can be a logo or symbol that visibly identifies the article as tagged.

In preferred methods, the drying time of the mark is less than 3 seconds.

Optionally, the methods of the invention further comprise analyzing the mark to determine the presence of the nucleic acid tag. In such embodiments, the analysis comprises amplifying the nucleic acid using PCR, and may further comprise probing the amplified nucleic acid with a specific labeled probe that hybridizes with the nucleic acid or sequencing the amplified nucleic acid. In some embodiments of the methods, the printed mark can be extracted or partially dissolved to isolate the nucleic acid tag for analysis.

In some embodiments, the article is a pharmaceutical or the packaging of a pharmaceutical, a cosmetic or the packaging of a cosmetic, an electronic article, subcomponent of an electronic article, or an assembly of electronic articles, packaging for an alcoholic beverage, and/or packaging for a tobacco product.

The invention also includes a system for applying a tagged ink composition to produce a mark on an article, comprising: an inkjet printer; an ink cartridge containing a nucleic acid tagged ink composition as described herein and incorporating a data chip that contains actual information, relational information or both regarding the specific nucleic acid tag contained in the ink. Optionally, the system comprises a data chip that, via direct contact between the inkjet printer and the data chip, provides confirmation of the identity of the nucleic acid tag present in the ink being printed. In some embodiments of this system, the ink cartridge further includes a radio frequency identification tag that contains data about the ink in the cartridge and the inkjet printer further includes a radio frequency identification tag reader that reads the data on the radio frequency identification tag. In addition, in some embodiments, the inkjet printer is designed to function and print only a predetermined tagged ink. In some embodiments, the inkjet printer and ink cartridge monitor data on ink batch and monitor ink usage of the printer to enable tracking and auditing of tagged ink consumption and logistical flow. In some embodiments, the system can provide validating data to a database confirming that an article has been marked with a specific nucleic acid tag.

The invention also comprises a method of production line printing using any of the tagged ink compositions described herein, comprising: advancing a plurality of like products past an inkjet printer including an ink reservoir with ink supplied to a print head, and marking each product with ink droplets generated by the inkjet printer as each product advances past the print head and according to a predetermined marking image.

### DETAILED DESCRIPTION

### 1. Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled reader should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount. Unless defined otherwise, all technical and scientific terms used herein have the same meaning as commonly understood by one of ordinary skill in the art.

The term “about,” as used herein, refers to a range of 10% on either end of the cited number. Therefore, for example, “about 100” indicates 100+/−10 or 90-110, while “about 50” indicates 50+/−5 or 45-55.

The terms “inkjet” or “ink jet,” as used herein, refer to inkjet printing, a type of non-contact printing that creates an image by propelling small droplets of ink onto a substrate such as paper, plastic, metal, glass, and the like. “Continuous inkjet” or “CIJ” methods are used, for example, in the marking and coding of products and packages. In this method, a pump directs a liquid ink composition from a reservoir to a nozzle to create a continuous stream of ink droplets, which are subjected to a controlled and variable electrostatic field, and thereby are charged as the droplets form according to the varying electrostatic field. The charged droplets are deflected to the proper location by passing through another electrostatic field to print the desired pattern on a substrate, or are recycled back to the reservoir for future use. An “inkjet ink” is any ink that is suitable for use in an inkjet printer.

The phrases “substantially free of” or “substantially no,” as used herein, in the context of a solvent or other component in the inventive ink composition, refers to a condition in which preferably no appreciable or readily detectable amount of the indicated component is present in the composition. “Substantially no” or “substantially free of” can refer to an amount which is below the detection limit of commonly used detection methods known in the art, or below the maximum amount permitted for the compound by regulation, or an amount below 5%, and preferably below 2%, below 1%, or below 0.5%. Preferably, the amount is below 1%.

The term “solvent,” as used herein, refers to a component whose primary function is to dissolve and carry the other components of the ink composition, and includes water and organic solvents such as alcohols (e.g., ethanol, methanol, and the like), ketones (e.g., acetone, methyl ethyl ketone (MEK), and the like), esters, amides, and ethers. The term “solvent” also refers to a mixture of solvents. Preferred solvents include MEK (also referred to as butanone, CH₃C(O)CH₂CH₃), acetone, methanol, ethanol, and any mixture thereof, which include less than 10% added water. A “volatile organic solvent” includes any organic solvent that has an evaporation rate of 0.1 or greater where butyl acetate=1.0. Volatile organic solvents should also exhibit a surface tension below about 0.030 N/m.

The term “solubilizing agent,” as used herein, refers to polar, protic solvents with a Hansen solubility parameter for hydrogen bonding δ_{H} of at least 10 MPa^{0.5}, such as dimethylsulfoxide, dimethylformamide, ammonia, alkyl amines (i.e., ethylamine, diethylamine, triethyl amine, and the like), alkanol amines (i.e., ethanol amine, triethanol amine, and the like), and alcohols (ethanol, methanol, 1,2-propanediol, 1,2-hexanediol, ethylene glycol, propylene glycol, glycerol, and the like).

The term “stabilizing agent,” as used herein, refers to cationic organic compounds such as a polymer, organic salt or surfactant with a quaternary ammonium group. It should be generally understood that stabilization via tonic interaction will not be effective without chemical separation and resulting metathesis of the normal cations associated with nucleic acids.

The term “nucleic acid,” as used herein, refers to any of the biopolymers or biooligomers composed of monomers (nucleotides), including polynucleotides (greater than about 20 nucleotides long) and shorter oligonucleotides (more than one and up to about 20 nucleotides long). The term includes single-stranded deoxyribonucleic acid (DNA), double-stranded DNA, single-stranded ribonucleic acid (RNA), double-stranded RNA, and the like. Nucleotides each contain a pentose sugar (ribose or deoxyribose), a phosphate group, and a nucleobase, also referred to as a “base.” The five commonly naturally-occurring nucleobases are adenine (A), cytosine (C), guanine (G), thymine (T), and uracil (U). Additional bases also found in nucleotides can include 5-methyl-cytosine, 5-hydroxymethylcytosine, methylcytidine, inosine, pseudouridine, dihydrouridine, 7-methylguanosine, xanthine, hypoxanthine, purine, 2,6-diaminopurine, and 6,8-diaminopurine. Preferably, the nucleic acids of the invention primarily (more than 95%) or substantially always (99-100%) contain only the five nucleobases A, C, G, T, and U. Preferably, the nucleic acid is double-stranded DNA and is at least ten base pairs in length and up to about 1000 base pairs. Natural and synthetic nucleic acids are contemplated for use with the invention.

An “amplifiable nucleic acid tag,” as used herein, refers to a tag for which the sequence is known so that a primer pair for amplification of the nucleic acid (or a portion of this nucleic acid) can be designed and prepared, or a tag where the sequence is not necessarily known by the user who is detecting and/or identifying the tag but for which a primer pair already exists that can amplify this tag, preferably specifically amplify this tag.

The term “Dalton,” as used herein, is a unit referring to atomic mass unit as a molecular weight. Therefore, a molecule with a molecular weight of 1000 atomic mass units, is said to have a molecular weight of 1000 Daltons or 1 kiloDalton, abbreviated 1000 Da or 1 kDa, respectively.

The term “colorant,” as used herein, refers to a dye, pigment or other substance that imparts color or modifies the hue of something else, and can refer to any such substance. Colorants include black dyes as well as other colors, and in some embodiments can be food grade, cosmetic grade or pharmacopeia grade colorants.

The term “code,” as used herein, refers to a printed mark which is decipherable in some form usually either visually or with automated optical recognition. Codes may include but are not limited to an alphanumeric string; a barcode of a public domain or proprietary format; a single dimensional (linear) or multidimensional (i.e., 2-D) barcode; a serialized data string; a random data string; a datamap derived or interpolated from an optical image; a human readable code; an encrypted code, and the like. A “code” is distinguished from a “mark,” which is any amount of printed or applied ink containing a nucleic acid tag which could range from singularly printed drops to simple printed shapes. In the most strict sense, a code is variable in nature and can be changed on a per product basis if needed. A “mark” may include a “code.”

The terms “covert” and “overt,” as used herein, refer to a status of printed marks on a product or its packaging. A “covert” mark is invisible to the naked eye or not easily discerned by the naked eye under normal lighting conditions. When referring to a code, a covert code may be invisible, or may be visible, but not readily recognized by a casual viewer as a code. For example, a code which is printed with an ink which is not visible to the naked eye, such as an invisible fluorescent code would be a “covert” code, while a printed series of numbers visible to the naked eye on the product would be an “overt” code. An example of an overt mark is a logo printed on an article or its packaging that has an ordinary appearance but also contains a DNA tag. An example of a covert mark is a smaller mark that is overprinted on top of an existing mark or printed without a colorant at all so that it is not noticeable but still functions as a tag. Thus, like a covert code, a mark printed with invisible ink which must be viewed and located under light of a certain wavelength in order to be detected is an exemplary covert mark. “Overt” indicates that the mark or code is discernable with the naked eye. A “hidden” mark is a mark that is printed inside the article or in a location not seen in ordinary use of the product.

The term “binder resin,” or “resin,” as used herein, refers to a substance that aids in making the ink composition stick to the substrate to which it is applied during printing. In general, a binder is a material that holds other materials together to form a cohesive whole or to impart adhesive properties, particularly onto nonporous or semi-porous substrates.

### 2. Overview

Nucleic acids such as DNA are a solution for providing tags that are unique, chemically and physically stable, difficult to copy, and straightforward to identify. DNA analysis methods have become so accurate that they have been adopted in most western countries' legal systems for unequivocal determination of origin of items. The most typical example of this use is as tags to determine the origin of stolen currency. The presence of a given DNA sequence on or inside of an article is equivalent to a unique identifier for a given type or owner of an article. The tag is defined by the sequence and identity of the base pairs contained in the DNA polymer backbone. A change in the sequence and identity of the base pairs in the DNA molecule provide a different DNA tag. Each unique DNA tag can be assigned to each individual customer or each product, and are easily changed over time, making it nearly impossible for others to copy. With these traits, DNA is a potential replacement for other less effective kinds of unique security tags.

However, the effectiveness of a polynucleotide tagging system is only as good as the means to deploy it, control it and deliver it to the product. Conventional screen or flexographic printing methods may use too much DNA to be practical. Also, it is difficult to use conventional printing methods as an add-on to existing printing processes to employ security tags, especially where manufactured articles are physically small or there are space constraints on a production line. Alternatively, using industrial inkjet or thermal transfer printers, for example, would be a good way to deploy this type of DNA-coded mark, but as of yet none have been successful.

A primary reason for this, overcome by the current invention, is that DNA, although it has a chemically stable backbone, is difficult to dissolve and stabilize in solution in conventional inks for inkjet printing. In some cases, ink solvents are not good solvents for DNA and the solubility limit is reached quickly. For some conventional detection methods, the amount of DNA required is simply too great to be stable in the medium. Another problem is that DNA is highly functionalized chemically, which can give rise to specific chemical incompatibilities with the components of the ink formulation which can cause precipitation of the DNA from the ink. It is vital that DNA be compatible with the complete ink formulation, namely the other ink components that have been successfully employed in the coding and marking industry. A further problem relates to the costs of manufacturing and purifying the unique nucleic acids which might lead to unsustainable prices of the tagged inks. Previously conceived DNA inks generally use an aqueous solvent which is slow-drying and do not comprise suitable resins and colorants that can provide durable marks with good adhesion, water resistance and print-wetting onto a variety of articles and are not compatible with the most preferred inkjet coding methodologies used in industrial settings.

Additionally, the systems of the current invention using a security tag ink compositions incorporating nucleic acids provide manufacturers with the ability to print at container or individual component level with high precision, high resolution and high reliability.

### 3. Results

Inks according to the invention include specific combinations of solvents, colorants and resinous ingredients and polynucleotide tags with specific molecular weights and concentrations that were found to be very stable. The DNA tags were reliably and unequivocally detectable by conventional PCR analysis methods in printed marks on various materials under various environmental conditions. They further showed good readability, dry time and adhesion when printed onto a variety of nonporous substrates which represented a range of manufactured goods.

Inks that were both visible and invisible or covert were demonstrated. A covert ink contained an additional UV luminescent dye. In addition to conventionally air-dried inks, an ink which can be thermally cured and cross linked was demonstrated which provided enhanced durability. The tagged inks were able to be printed using an industrial inkjet printer for coding products. A series of reliability tests were also conducted to show that the added tags did not impact printer reliability and were stable enough to be detected again by PCR after operation in the printer and after simulated bottle shelf-life studies. As such the invention overcomes barriers with the aforementioned prior art.

### 4. Embodiments of the Invention

A. Introduction

Nucleic acid tags in a printed mark can be extracted and analyzed by a number of methods. The tag remains secure as long as it cannot be isolated in great enough quantity to be sequenced and identified. Often a first approach that counterfeiters use to defeat a security feature like a printed mark is simply re-use the mark and apply it to other unauthorized products. This can be done by reusing the packaging or by isolating the security component within a tagged region and applying it to new products. Therefore, if too great a quantity of a security tag is applied to individual items, it will be possible to extract and re-cycle the unique identifier in the security tag. Thus, for any security tag, it is important to provide as little as possible in the ink (while still providing enough for detection) and to limit the potential distribution of any inks that contain tags.

An inherent weakness of many systems that employ tagged inks is the inability to deter simple theft, i.e., methods to monitor and protect the tagged ink itself from distribution beyond what is intended. This is especially important if the printing does not occur centrally, but perhaps at many various production nodes or different physical addresses. Hence, there exists a need for systems that can enable validation and tracking of polynucleotide-containing inks to deter would-be thieves and to help confirm to producers that their unique security solution is not being replicated in any fashion.

Nucleic acids are still relatively expensive. To be an effective unique identifier, the DNA strand must have sufficiently different individual base pair sequences compared to other similar tags. For the sake of printer compatibility, DNA tags also must exhibit chemical characteristics like a suitably compatible molecular weight. These attributes necessarily increase the tag's production difficulty and cost.

In order to enable the widespread use of polynucleotides as security marks, a printing method must also be a reasonably good dosing method. Hence, there is also a need for printing methods which apply just enough of the active polynucleotide tag to make it impossible to detect above environmental noise (using the most sensitive analysis methods) and to limit the potential for re-use where they are not intended.

In many cases an overt code which may or may not contain serialized data is a convenient and secure means to deliver a tag. These features can enhance the overall deterrence and effectiveness of the security tag. In some cases, it is preferable to hide the printed mark. However, hidden printed marks are difficult to apply in practice in small areas on products or packaging. Therefore, in some cases a better alternative is to deliver an invisible or covert code which is either compatible with what is already present on the product surface or which is relatively invisible and difficult to detect.

The invention is most effective when combined with state-of-the-art but conventional polymerase chain reaction (PCR) methodology. PCR is a technique used in molecular biology to amplify a single copy or a few copies of a particular DNA across several orders of magnitude, generating over a million copies in just 20 cycles, as the concentration doubles every cycle. The DNA cannot be easily amplified without an appropriate specific primer that hybridizes to the DNA to be amplified (based on the specific sequence of the DNA tag). During carefully staged PCR procedures that are unique to a given DNA tag and ink combination, the previously known tag can be amplified to sufficient numbers to reach the detection threshold. In order to isolate detectable amounts of DNA without knowing the sequence or having suitable primers ahead of time, at least a million marks containing the tag might need to be collected and extracted before assay, an exercise that would be futile in most cases to even the most ambitious counterfeiters. The presence of the tag can be proven with near 100% assurance due to a combination of the specific application of the PCR amplification process and the statistical unlikelihood of amplifying impurities which are similar to the target amplicon and the great improbability of the presence of impurities highly similar to the tag.

B. Ink Compositions

1. General Considerations

In general, an ink jet ink composition must meet strict physical and chemical properties requirements to be compatible with ink jet printing systems. Further, the ink must be quick drying and smear resistant, and be capable of passing through the ink jet nozzle(s) and printer system filters without clogging, and permit rapid cleanup of the machine components with minimum effort. The selection of fast drying, durable polymers for inkjet inks requires both a good theoretical understanding of these properties as well as empirical validation of their performance. After drying/curing of the printed mark, the mark should be sturdy enough to resist removal at least by ordinary use of the product or packaging, but in some cases the marks must pass specific durability tests.

Inks of the present invention exhibit properties that allow for good operation in inkjet printers or thermal transfer (overprinters; TTO) printers. When used for inkjet printing, the inks exhibit a suitably short drying time (less than about 5 seconds and preferably less than about 3 seconds), and compatible viscosity, surface tension, conductivity, solids content, and sonic velocity.

The dry time of the inkjet composition preferably is short enough to enable maximum flexibility when integrated into production. For example, the dry time is preferably about 5 seconds or less and more preferably about 3 seconds or less when printing ink drops at about 1 nanoliter in volume with a resolution between about 50 and 100 dpi at an ambient temperature of about 25° C. at a relative humidity of about 50%.

The inkjet composition at jetting temperature preferably has a viscosity between 1 and 30 cPs, preferably between 1.5 and 15.0 cPs, and most preferably between 2.5 and 12.0 cPs. The ink composition preferably has a surface tension as measured by the bubble-tensiometer method at 25° C. of about 20 mNm⁻¹ to about 50 mNm⁻¹, preferably of about 21 mNm⁻¹ to about 40 mN m⁻¹, or more preferably from about 22 mNm⁻¹ to about 30 mN m⁻¹. The solids content of the ink composition at 25° C. is equal to or less than 100% by weight, preferably less than 50% by weight, more preferably less than 40% by weight, and most preferably less than 30% by weight. In some embodiments, the resulting sonic velocity of the ink preferably is between 1100 and 1600 meters per second as measured by the acoustic method.

In a continuous inkjet printer, ink is subjected to rapid movement through narrow passageways (i.e, micro-sized nozzle and pump gears), droplet formation, increased temperature, and repeated filtration as it circulates through the printer during use. The nucleic acid tag in the ink therefore also is subjected to all these conditions, while dispersed or dissolved in an organic solvent environment. DNA is known to be soluble in an aqueous solution, but to precipitate in an organic or non-polar environment and to degrade under conditions of heat and shear forces. It is surprising that the ink compositions of the invention would be stable and produce a fast-drying mark from which the DNA can be extracted and identified reliably.

Due to the stable inherent nature of nucleic acid tags contained in the ink, once the tag is delivered the tag is detectable even after for storage periods and harsh exposure conditions to light, oxidative conditions, etc. up to and including the product's life cycle. Embodiments of the invention are stable and the nucleic acid is detectable in a printed mark after bottle storage for at least 1 year and during operation within a continuous inkjet printer for more than 900 hours.

2. Solvents

The inkjet ink compositions according to this invention can include any suitable volatile organic solvent or solvent system (combination of solvents) as the carrier. A volatile organic solvent is one that has a relative evaporation rate greater than or equal to 0.1 where n-butyl acetate has an evaporation rate of 1.0. In some embodiments, the jet ink composition preferably is free or substantially free of slow evaporating solvents, for example, solvents having an evaporation rate of less than 0.1 relative to n-butyl acetate or a boiling point greater than 160° C. at standard conditions. If a slow evaporating solvent is present, it is preferably present in a small quantity, for example, about 5% by weight or less, more preferably about 3% by weight or less, and even more preferably about 1% by weight or less, of the jet ink composition.

Suitable organic solvents contemplated for use in the invention include alcohols, ketones, esters, ethers, amides, and mixtures thereof. The organic solvents are preferably selected from C1-C4 alcohols, C3-C6 ketones, and mixtures thereof. Examples of suitable C1-C4 alcohols include methanol, ethanol, 1-propanol, 2-propanol, 1-butanol, 2-butanol, and 2-propanol. Examples of C3-C6 ketones include acetone, methyl ethyl ketone (MEK), methyl n-propyl ketone, methyl isopropyl ketone, diethyl ketone, methyl n-butyl ketone, methyl isobutyl ketone and cyclohexanone. Examples of ethers include diethyl ether, dipropyl ether, dibutyl ether, tetrahydrofuran, propylene glycol methyl ether, and diethylene glycol monoethyl ether. Examples of esters include methyl acetate, ethyl acetate, n-propyl acetate, isopropyl acetate, and n-butyl acetate. Preferred primary solvents for inkjet applications are methyl ethyl ketone (MEK), acetone, methanol, ethanol and any combination or mixture of two or more of any of these primary solvents. The solvent or solvent mixture should be selected by the practitioner based on the solubility of the ink components, the overall target ink drying rate, and also the solubility of the nucleic acid contained in the ink.

The solvent can comprise a ‘solubilizing agent.’ In general, solvents which are suitable for nucleic acids tend to be poor solvents for the necessary dyes and resins which impart suitable adhesion and fastness properties to the printed code. In terms of three-dimensional Hansen solubility parameters, DNA is typically assigned values of δ_{D}=19.0; δ_{P}=20.0; and δ_{H}=11.0 where δ_{H} represents the energy of hydrogen bonding and δ_{P} represents the energy from dipole interactions. A solvent exhibiting particularly low δ_{H} or δ_{P} values can render a polynucleotide insoluble even if that solvent is used as a partial solvent in a blend of solvents. However, a solubilizing agent chosen with the right Hansen parameters can positively impact solution stability of the nucleic acid. In some cases it is preferred that a solubilizing agent is employed with a δ_{H} that is ≥10. In other cases it is more preferred that δ_{H} is ≥10 and <24 and also that δₚ is ≥10. Preferred alcohols with suitable δₚ and δ_{H} parameters are monohydric alcohols (e.g., ethanol, methanol, etc.) but alcohols could also or alternatively be dihydric (e.g., 1,2-propanediol; 1,2-hexanediol, etc.) or trihydric (e.g., ethylene glycol, propylene glycol; glycerol, etc.).

In some embodiments, other solubilizing agents including dimethylsulfoxide, formamide, dimethylformamide, ammonia, alkyl amines (i.e., ethylamine, diethylamine, triethyl amine etc.), alkanol amines (i.e., ethanol amine, triethanol amine, etc.), benzyl alcohol, cyclohexanol, acetic acid, polyethyelene glycol, polypropylene glycol or urea can be included at appropriate levels while limiting impact to other aforementioned aspects of ink performance. The best solubilizing agent should also, understandably, be selected based on their melting point (<−10° C.), boiling point (>50° C.), low odor, low surface tension (<0.040 N/m) and relative health and safety profile.

In some cases, the solubility or stability of the nucleic acid tag can be enhanced by the use of a ‘stabilizing agent’ which is an organic, cationic substance that can interact ionically with the anionic groups on a polynucleotide chain. Stabilizing agents, however, are not required to achieve necessary stability according to the current inventive formulas.

In some embodiments, water may be a good co-solvent choice because DNA is soluble in water, but water cannot be used at levels which are too high because the desirable resins and colorants exhibit extremely low solubility in water and water exhibits a very high surface tension which makes it difficult to design inks that sufficiently wet-out nonporous substrates.

Solvents and solvent mixtures useful in inks according to some of the embodiments of the invention also optionally contain up to about 10% added water, preferably less than about 10% added water, more preferably less than about 8% added water, more preferably less than about 6% added water, more preferably less than about 5% added water, more preferably less than about 4% added water, more preferably less than about 2% added water, and most preferably less than about 1% added water. Compositions can contain, for example, about 5% added water, about 1% added water, about 0.5% added water, about 0.2% added water or about 0.1% added water. Preferred ink compositions contain less than 10%, 5%, 2%, or 1% total water. In some embodiments, the ink composition contains no added water. It is understood that some residual water (or even detectable water) may be contained in one or more of the components of the ink or that water may come into the composition during manufacture, however it is preferable that this be minimized so that the amount of water in the final composition is controlled. This water, which can be an incidental or purposeful minor component of some of the other components in the ink, is distinguished from “added water,” which is deliberately added as a separate component. For example, the nucleic acid tag component of an ink can be provided in an aqueous solution when mixing the ink, but the volume added is very small and preferably the water in this component does not affect the total amount of water in the composition appreciably or measurably, thus would not be considered “added water.”

Any suitable amount of ink carrier can be used depending on the printing method to be used so long as the nucleic acid tag is substantially soluble or dispersed within the ink composition. Typically the carrier (solvent) is used in an amount of up to about 95%, preferably in an amount of from about 35% weight to about 90% by weight, and more preferably in an amount of from about 65% by weight to about 85% weight of the ink composition. Preferred ink compositions contain about 40%, about 45%, about 50%, about 55%, about 60%, about 70%, about 75%, or about 80% solvent or carrier, for example. For TTO dry inks, the ink can be completely free of solvents and contain only waxes or resinous materials in the formulation.

The solvent or solvent system chosen in any particular embodiment is selected based on the drying time of the ink composition, which preferably is less than 3 seconds. Therefore, the solvent or mixture of solvents preferably has an evaporation rate of greater than 0.01, preferably greater than 0.1.

3. Colorants

The ink composition can include any suitable colorant or colorants, which may be a dye or a pigment, or a combination of dyes or pigments. In some embodiments, one or more dyes are employed as the colorant. Preferred colorants include one or more dyes selected from the group consisting of acid dyes, basic dyes, solvent dyes, disperse dyes, mordant dyes, reactive dyes and any combination thereof. Examples of solvent dyes include naphthol dyes, azo dyes, metal complex dyes, anthraquinone dyes, quinoimine dyes, indigoid dyes, benzoquinone dyes, carbonium dyes, naphthoquinone dyes, naphthalimide dyes, phthalocyanine dyes, nigrosine dyes and perylene dyes.

For example, the thermal ink jet ink composition can include one or more dyes selected from the group consisting of C.I. Solvent Yellow 19, C.I. Solvent Yellow 21, C.I. Solvent Yellow 61, C.I. Solvent Yellow 80, C.I. Solvent Orange 1, C.I. Orange 37, C.I. Orange 40, C.I. Solvent Orange 54, C.I. Solvent Orange 63, C.I. Solvent Red 8, Solvent Red 49, C.I. Solvent Red 81, C.I. Solvent Red 82, C.I. Solvent Red 84, C.I. Solvent Red 100, C.I. Acid Red 92, C. I. Reactive Red 31, Orient Pink 312, C.I. Basic Violet 3, C.I. Basic Violet 4, C.I. Solvent Violet 8, C.I. Solvent Violet 21, C.I. Solvent Blue 2, C.I. Solvent Blue 5, C.I. Solvent Blue 11, C.I. Solvent Blue 25, C.I. Solvent Blue 36, C.I. Solvent Blue 38, C.I. Solvent Blue 55; C.I. Solvent Blue 70, C.I. Solvent Green 3, C.I. Solvent Black 3, C.I. Solvent Black 5, C.I. Solvent Black 7, C.I. Solvent Black 22, C.I. Solvent Black 26, C.I. Solvent Black 27, C.I. Solvent Black 29 (VALIFAST BLACK 3808 or ORASOL BLACK X-55), C.I. Acid Black 123, C.I. Solvent Black 48 (MORFAST BLACK 101™), C.I. Oil Blue 613, and any combination thereof, and preferably one or more dyes selected from the group consisting of C.I. Solvent Black 29 (ORASOL BLACK RLI), C.I. Solvent Black 27, C.I. Solvent Black 48, C.I. Solvent Black 3 (Oil Black 860), C.I. Basic Violet 3, C.I. Solvent Blue 38, C.I. Solvent Blue 70, C.I. Oil Blue 613, C.I. Solvent Red 49 (ORIENT PINK™ 312), C.I. Solvent Orange 54 (VALIFAST ORANGE™ 3210), and any combination thereof.

Any suitable pigment can be used, for example, one or more pigments selected from the group consisting of phthalocyanine blue, carbon black, mars black, quinacridone magenta, ivory black, prussian blue, cobalt blue, ultramarine blue, manganese blue, cerulean blue, indathrone blue, chromium oxide, iron oxides, viridian, cobalt green, terre verte, nickel azo yellow, light green oxide, phthalocyanine green-chlorinated copper phthalocyanine, burnt sienna, perinone orange, irgazin orange, quinacridone magenta, cobalt violet, ultramarine violet, manganese violet, dioxazine violet, zinc white, titanium white, flake white, aluminum hydrate, blanc fixe, china clay, lithophone, arylide yellow G, arylide yellow 10G, barium chromate, chrome yellow, chrome lemon, zinc yellow, cadmium yellow, aureolin, naples yellow, nickel titanate, arylide yellow GX, isoindolinone yellow, flavanthrone yellow, yellow ochre, chromophthal yellow 8GN, toluidine red, quinacridone red, permanent crimson, rose madder, alizarin crimson, vermilion, cadmium red, permanent red FRG, brominated anthranthrone, naphthol carbamide, pervlene red, quinacridone red, chromophthal red BRN, chromophthal scarlet R, aluminum oxide, bismuth oxide, cadmium oxide, chromium oxide, cobalt oxide, copper oxide, iridium oxide, lead oxide, manganese oxide, nickel oxide, rutile, silicon oxide, silver oxide, tin oxide, titanium oxide, vanadium oxide, zinc oxide, zirconium oxide, and any combination thereof.

In some embodiments, the pigments are selected from the group consisting of azo pigments, phthalocyanine pigments, quinacridone pigments, dioxazine pigments, isoindolinone pigments, metal oxide pigments, carbon black, and any combination thereof. The pigments can have any suitable particle size, for example, from about 0.005 micron to about 15 microns, preferably from about 0.005 to about 1 micron, and more preferably from about 0.01 to about 0.3 micron.

In any of the embodiments, the visible colorant can be present in an amount from about 0.01% to about 10%, preferably from about 0.5% to about 7%, and more preferably from about 1% to about 5% by weight of the ink composition.

In certain embodiments, the ink contains a luminescent compound or dye, either alone, or in addition to another colorant. A luminescent compound or dye is any compound that is soluble in the carrier solvent to an extent that provides measurable fluorescence in solution and to an extent characterized by a weight formulation percentage that is greater than 0.01%. Luminescent compounds may be fluorescent, phosphorescent, bioluminescent, or the like, and are selected from the following general classes aromatic (e.g., anthracene); substituted aromatic (e.g., nitrobenzene); heterocyclic (e.g., furan, thiophene); cyanine; phthalocyanine; naphthalocyanine; xanthene (e.g., fluorescein, rhodamine), acridine (e.g., euchrysine); phenazine (e.g., safranin), napthol; porphyrin; coumarin; pyrromethene; oxazine; oxazole (e.g., benzooxazole), perylene, napthalimide, triazine, imidazoline, di/triazole, stilbene (e.g., biphenystilbene) and any combination thereof. Applicable luminescent compounds are any that possess a luminescence emission peak wavelength between 400 and 750 nm.

The colorants in the inks according to the invention can be visible to the naked eye and leave a visible mark after printing. However in certain embodiments, the ink composition includes one or more invisible luminescent compounds as the sole colorant, rendering the ink and the printed mark not visible to the naked eye under normal illumination, or one or more invisible luminescent compounds in combination with a visible colorant. Particularly suitable kinds of invisible fluorescent compounds are optical brighteners, including oil soluble varieties such as benzoxazoles. One specific example is 2,2′-(2,5-thiophenediyebis[5-tert-butylbenzoxazole] (CAS no. 7128-64-5; sold as Uvitex OB™ and Tinopal OB™). This compound is often used as a tracer compound in oily media (such as fuels, pesticides, etc.) because it is hydrophobic and possesses good solubility in selected polar organic solvents such as MEK.

A particularly suitable xanthene fluorescent dye for use as a luminescent compound conforms to the structure for C.I. Index Basic Red 11:1 and is sold under the trade name Basonyl Red 560™. Another suitable dye example is C.I. Index Solvent Red 49. Other preferred luminescent compounds include fluorescent naphthalimide and perylene dyes sold under the trade name Lumogen™ from BASF® Corporation. One particularly suitable example is Perylene F Red™ 300 (or 305). Other examples of preferred Perylene dyes are trade named Lumogen F Yellow 083™, Lumogen F Yellow 170™, Lumogen F Orange 240™, Lumogen F Pink 285™, Lumogen F Violet 570™, and Lumogen F Blue 650™.

In any of the embodiments, the luminescent compound, if present, is present in an amount from about 0.001% to about 10%, preferably from about 0.05% to about 3%, and more preferably from about 0.1% to about 2% by weight of the ink composition.

In some embodiments of the invention, it is desirable to produce marks on food, cosmetic, or drug products, or their packaging. Therefore, any particular resins that are approved in different jurisdictions for use on these types of products can be used in the ink compositions according to the invention. Because different countries or regions have different specific lists of ingredients that are suitable for food, cosmetic or pharmaceutical printing, the following lists of these type of colorants is noninclusive. Other colorants can be chosen by any person of skill.

Other useful colorants therefore include food grade, cosmetic grade or pharmacopeia grade colorants. For example, dyes or pigments characterized as FD&C or D&C grade (i.e., those specified by the e-CFR Title 21, part 74 regulations) in the U.S or their foreign variants (i.e., the European Union E-List and the UN FAO color list) are printing directly onto food and in many circumstance onto cosmetic or drug usage. Examples include but are not limited to FD&C Blue 1 (C.I. 42090:2), FD&C Blue No. 2, C.I. Food Blue 5, D&C Blue 4, D&C Blue 9, BLUE VRS, Brilliant Blue FCF, Patent Blue 5, FD&C Red 3, FD&C Red 4, FD&C Red 40, D&C Red 6 (C.I. 15850), D&C Red 7 (C.I. 15850:1), D&C Red 9 (C.I. 15585:1), D&C Red No. 17, D&C Red 21 (C.I. 45380:2), D&C Red 22 (C.I. 45380:3), D&C Red 27 (C.I. 45410:1), D&C Red 28 (C.I. 45410-2), D&C Red 30 (C.I. 73360), D&C Red 31, D&C Red 33 (C.I. 17200), D&C Red 34 (C.I. 15880:1), D&C Red 36, D&C Red 39, C.I. Food Red 7, Altura Red AC, Sudan Red G, Cirus Red 2, Fast Red E. Red 2G, Red 10B, Rhodamine B, Scarlet GN Ponceau 2R/4R/6R/SX, D&C Violet-2, methyl violet, Violet 5BN, Acid Fuchsin B, Benzyl Violet 4b, indigotine, FD&C Yellow 5 (C.I. 19140:1), FD&C Yellow 6 (C.I. 15985:1), FD&C Yellow 10 (C.I. 47005:1), D&C Yellow 7, D&C Yellow 8, D&C Yellow 10, D&C Yellow 11, C.I. Food Yellow 3, C.I. Food Yellow 23, chryosoine yellow, Fast Yellow AM, Napthol Yellow S, Sunset Yellow FCF, Yellow 2G, Yellow 27175N, D&C Orange 4, D&C Orange 5 (C.I. 45370:2), D&C Orange 10, D&C Orange 11, Orange B, D&C Green 5, Orange G, Orange GN, Orange I, Orange RN, Sudan G, D&C Green 6, D&C Green 8, FD&C Green 3, Green S, Fast Green FCF, Guinea Green B, Light Green SF, D&C Brown 1, Brown FK, Brown HT, D&C Black 2, D&C Black 3, Black 7984, and Brilliant Black PN. Especially preferred dyes from this list are FD&C Blue 1, FD&C Red 3, FD&C Red 40, FD&C Yellow 5, FD&C Yellow 6, FD&C Green 3 or titanium dioxide. The most useful food grade dyes are inherently soluble in organic solvents with up to about 10% water content in the ink, such as FD&C Red 3.

Natural colorants obtained from plants or extracts of insects, are also suitable, either in pigment form or as organic solvent soluble extracts. Examples of natural colorants include beta-carotene, annatto extract, astaxanthin, astaxanthin dimethyldisuccinate, dehydrated beets, cholorophyllin (and its copper complexes and various salts), ultramarine blue, caramel, canthaxanthine, β-Apo-8′-carotenal. β-Carotene or its derivatives, cochineal extract, grape (or skin) extract, guanine, fruit juice, vegetable juice, carrot oil, corn endosperm oil, paprika, paprika oleoresin, saffron, tomato extract (lycopene), turmeric (curcumin), amaranth, anthocyanins, azorubine, bixin, blackcurrent extract, canthaxanthin, citraxanthin, carmine, carthamus red, carthamus yellow, eosine, erythrosine, orcein/orchil, paprika extract, quercetin, persian berries, riboflavin, tagetes extract, tartrazine, ultramarines, and xanthophyll.

4 Resins

Ink compositions according to the invention optionally contain at least one binder resin to provide both the desired properties for functioning in an inkjet printer, and sufficient adhesion so that the printed marks remain attached to the market object for as long as required by the application without transfer of the printed marks to another surface that may come in contact with the printed object after marking. In addition to the primary binder resin, one or more co-resins optionally are employed. In some embodiments, however, particularly if the mark is designed to be absorbed into or becomes part of the article to be printed, a resin is not necessary for printing onto a porous surface.

Any suitable combination of resins as known in the art can be used. More specifically, any thermoplastic resin can be employed that is soluble in the ink solvents, is sufficiently hard at room temperature (softening point>25° C.) and that exhibits a molecular weight under about 100 kDa. In certain embodiments, the ink composition includes one or more resins, which preferably are selected from acrylic resins, vinyl chloride/vinyl acetate copolymers, polyesters, polyvinyl butyral resins, epoxy-phenolic resins, functionalized silicone resins, cellulose acetate propionate resins, polyurethane resins, modified rosin resins, phenolic resins, polyamide resins, ethyl cellulose resins, cellulose ether resins, cellulose nitrate resins, polymaleic anhydride resins, acetal polymers, styrene/methacrylate copolymers, aldehyde resins, copolymers of styrene and allyl alcohols, epoxies, polyhydroxystyrenes, ketone resins, polyketone resins, sulfonamide-modified epoxy resins, terpene phenolic resins, and any combination thereof. Preferred resins are styrene acrylic resin and nitrocellulose resin. Additionally, in other embodiments, it is preferred that the thermoplastic resins chosen are cross-linkable or curable by exposure to high temperatures (e.g., within a thermal oven) or by light irradiation (e.g., under a UV lamp). In yet further embodiments, the resins, including the main binder resin, can comprise a liquid resin(s) instead of a solid resin(s) at room temperature and below. Such binders must be cured or cross-linked immediately after printing by at least one of the methods described above in order to achieve permanence on the surface.

Preferred specific examples of binder resins for use with the invention include cellulose esters such as cellulose acetate butyrate resin and cellulose acetate propionate resin. Particularly suitable fixative resins are ones that have molecular weights (MW) between about 20,000 Da and 120,000 Da and glass transition temperatures (T_{g}) between 70° C. and 180° C.

Suitable acrylic resins can be homopolymers or incorporate two or more monomers with or without specific functional groups. Functionalized acrylic resins may be derived from an alkyl-type monomer such as a methacrylate plus a functionalized monomer such as acrylic acid or methacrylic acid; basic monomers such as amino acrylates; or neutral functional monomers that contain hydroxyl groups. Examples of suitable resins are those from Dow Chemical® Corporation sold under the trade-name Acryloid™ or Paraloid™ or Dianal™ resins from Dianal® Corporation. A specific example of a non-functionalized resin is sold under the trade name B-60 which is a methylmethacrylate and butylmethacrylate copolymer with a molecular weight of approximately 50,000 Da. A specific example of a preferred functionalized acrylic resin is Dianal™ PB-204. Other preferred resins are ones that incorporate pendant amine groups as is disclosed in U.S. Pat. No. 4,892,775. Examples of acrylic resins also include styrene-acrylic resins which can be made by copolymerizing styrene with acrylic monomers such as acrylic acid or methacryl acid, and optionally with alkyl acrylate monomers such as methyl acrylate, methyl methacrylate, butyl acrylate, butyl methacrylate, hydroxyethyl acrylate, hydroxyethyl methacrylate, and the like made by BASF®, under the trade name JONCRYL™. Examples of JONCRYL™ resins include JONCRYL™ 555, 586, 678, 680, 682, 683, and 67.

Examples of vinyl acetate/vinyl chloride copolymers include those under the trade name of Vinnol™ from Wacker Chemie®, Inc. These might include structurally modified carboxyl-vinyl chloride/vinyl acetate polymers such as Vinnol E15/45M, hydroxyl-modified vinyl chloride/vinyl acetate polymers such as Vinnol™ E15/40 A or unmodified vinyl chloride/vinyl acetate polymers such as Vinnol™ H14/36. Vinyl acetate/vinyl chloride copolymers with any structural modifications or ratio of vinyl choride:vinyl acetate may be employed, as long as they are soluble in the carrier. Examples of polyvinyl butyral resins are PIOLOFORM™ BN18, available from Wacker Chemie® AG, and MO WITAL™ B20H available from Kuraray America®, Inc. Examples of ethyl cellulose resins are Ethocel available from Dow Chemical®.

Suitable rosin esters include gum rosins, wood rosins or tall oil rosins or modified versions thereof. Hydrogenated forms are generally preferred due to their relative stability. General examples of hydrogenated rosin esters include those from Arizona Chemical® (Uni-tac™), Eastman Chemical® (Foral™, Staybelite™, Pentalyn-H™); and, Arakawa Chemical® (Superester). Non-hydrogenated varieties are suitable including those from Eastman® (Pentalyn™, Pexalyn™, etc.); Arakawa® (Pencel™, etc.); and, Arizona Chemical® (Sylvateac™, Sylvalite™, etc.). Preferred specific examples include low acid number, apolar varieties such as STAYBELITE™ ESTER 10, available from Eastman Chemical®, Inc., and Superester™ A-75 from Arakawa Specialty Chemicals®, Inc. An example of a preferred wood rosin ester resins is UNIREZ™ 8115, available as a 40% solution in ethanol from Penn Color®, Doylestown, Pa., which is a hydrogenated wood rosin ester. Examples of cellulose nitrate resins are NOBEL™ DLX 3-5 or NOBEL™ DHX 5-8, available from Nobel Enterprises®. Examples of polyvinyl butyral resins are PIOLOFORM™ BN18, available from Wacker Chemie® AG, and MOWITAL™ B20H available from Kuraray America®, Inc. Examples of acrylic and styrene/acrylic resins are Joncryl 611, 682, and 586 (available from BASF®, USA); DM-55™, Paraloid™ B-66, and B-72 (available from Dow Chemical®, USA); and Elvacite™ 2013 and 4055 (available from Lucite® Inc.). Examples of vinyl resins include UCAR VYHH, VMCH, VMCA, and VAGF (available from Dow Chemical® Company, USA; equivalent replacement resins from other suppliers) and Vinnol™ E15/45, E15/40A, H14/36, E15/45M, and E16/40A (available from Wacker Chemie® AG, Germany). Examples of polyhydroxystyrene resins include poly(p-hydroxystyrene) from DuPont®. An example of a sulfonamide-modified epoxy resin is AD-PRO MTS™, available from Rit-Chem®. Examples of sulfonamide-modified formaldehyde resins are P-TOLUENE SULFONAMIDE FORMALDEHYDE RESIN™, available from Jiaxing Chenlong Chemical Company, Ltd. and RIT-O-LITE™ MHP, available from Rit-Chem®. An example of a suitable polyamide resin is ARIZONA 201-150™ available from Arizona Chemical Company®, Jacksonville, Fla., or COGNIS VERSAMID 756™, available from Cognis GmbH, Monheim am Rhein, Germany, both of which are alcohol-soluble polyamide resins.

In some embodiments, suitable hinder resins include those that are both compatible with the inkjet process and considered food grade or pharmacopeia grade. Suitable resins of this type include but are not limited to shellac, modified cellulosic resin (i.e., ethyl cellulose or hydroxypropyl cellulose), and polyvinylpyrrolidone. For example, shellac offers excellent print image adhesion and rub resistance. Inkjet compatible grades of shellac generally exhibit a molecular weight which is less than about 10 kDa. Any suitable shellac can be used, as determined by the practitioner and depending on the use and the substrate. Preferably, a refined bleached shellac is used to prepare the ink concentrate of the present invention. The term “Refined” refers to the removal of the natural shellac wax, and thus a refined bleached shellac may have a low wax content of about 0.1-0.2% by weight, whereas an unrefined bleached shellac may have a wax content of 4.0-5.5% by weight.

The combined binder and co-resins may be present in any suitable amount, for example, in an amount from about 0% to about 30%, preferably from about 1% to about 15%, and more preferably from about 2% to about 12% of the ink composition.

In application where a particularly durable mark is required, it is preferable to use an ink that can be cured or cross-linked. Such inks after exposure to heat can be rendered insoluble in most or all solvents without the need for a protective top coat. In such cases, the nucleic acid tag is present, but may be more difficult to extract for purposes of authentication.

5. Other Agents

In still other embodiments, a secondary coating can be applied over the printed mark containing either thermoplastic resins, thermosetting resins or UV curable resins in order to protect the printed mark from dissolution or abrasion and render it more permanent.

Ink compositions for the continuous inkjet process should exhibit solution conductivities greater than 200 μSiemens; and more preferably greater than 500 μSiemens, and therefore optionally include a conductive agent (an ionic species added to the ink composition to impart measurable conductivity). Preferred conductive agents are cation/anion pairs (salts). Preferably the cations are alkali earth metals, alkali metals (i.e., Li⁺, Na⁺, K⁺), ammonium, alkyl/aryl ammonium and alkyl/aryl phosphonium and the like. Typical anions for the cation/anion pairs are halides, halo-phosphates (e.g., hexofluorophosphate), halo-antimonates, halo-borates, phenyl borates, nitrates, phosphates, sulfates, phosphonates, sulfonates, carbonates, carboxylates, thiocyanates, acetates, triflates; tosylates and the like. Conductive agents are typically only added to impart just enough electrical conductivity for use in inkjet printing to ink compositions to be used in inkjet printing. In a typical such ink composition, conductive agents are provided in an amount from 0.1 to 2.5% by weight. In some formulations the colorant can function as the conductive agent.

The ink composition also optionally can further include one or more additives such as plasticizers, surfactants, defoamers, humectants, adhesion promoters, and mixtures thereof. Suitable plasticizers can be polymeric and can be added in addition to a binder resin. Plasticizers generally have molecular weights that are less than 5,000 amu. Examples of suitable plasticizers include phthalate plasticizers, e.g., alkyl benzyl phthalates, butyl benzyl phthalate, dioctyl phthalate, diisobutyl phthalate, dicyclohexyl phthalate, diethyl phthalate, dimethyl isophthalate, dibutyl phthalate, and dimethyl phthalate, esters such as di-(2-ethylhexy)-adipate, diisobutyl adipate, glycerol tribenzoate, sucrose benzoate, dibutyl sebacate, dibutyl maleate, polypropylene glycol dibenzoate, neopentyl glycol dibenzoate, dibutyl sebacate, and tri-n-hexyltrimellitate, phosphates such as tricresyl phosphate, dibutyl phosphate, triethyl citrate, tributyl citrate, acetyl tri-n-butyl citrate, polyurethanes, acrylic polymers, lactates, oxidized oils such as epoxidized soybean oil, oxidized linseed oil, and sulfonamide plasticizers such as Plasticizer 8.

When present, the plasticizer preferably is present in an amount from about 0.01% to about 5.0%, preferably from about 0.1% to about 2.5%, and more preferably from about 0.25% to about 1.0% by weight of the ink composition.

Examples of surfactants include siloxanes, silicones, silanols, polyoxyalkyleneamines, propoxylated (poly(oxypropylene)) diamines, alkyl ether amines, nonyl phenol ethoxylates, ethoxylated fatty amines, quaternized copolymers of vinylpyrrolidone and dimethyl aminoethyl methacrylate, alkoxylated ethylenediamines, polyethylene oxides, polyoxyalkylene polyalkylene polyamines amines, polyoxyalkylene polyalkylene polyimines, alkyl phosphate ethoxylate mixtures, polyoxyalkylene derivatives of propylene glycol, and polyoxyethylated fatty alcohols, fluorinated surfactants. A specific example of a suitable polymeric surfactant is Silicone Fluid SF-69 which is a blend of silanols and cyclic silicones. A specific example of a siloxane polyalkyleneoxide copolymer surfactants includes SILWET™ L-7622.

In any of the embodiments, the surfactant additive, when present, preferably is present in an amount from about 0.001% to about 2.0% by weight and more preferably from about 0.005% to about 0.5% by weight of the ink composition.

The thermal ink jet ink composition optionally also includes additional ingredients such as bactericides, fungicides, algicides, sequestering agents, buffering agents, corrosion inhibitors, antioxidants, light stabilizers, anti-curl agents, thickeners, dispersing agents, and other agents known in the art of ink compositions. In a preferred embodiment, the ink composition is free or substantially free of antioxidants.

The ink compositions of the invention preferably are free of or substantially free of stabilizing agents and solubilizing agents.

C. Nucleic Acid Tags

Suitable nucleic acid tags according to the invention are double-stranded DNA, single-stranded DNA, double-stranded RNA, and single-stranded RNA. Such nucleic acids are well known and any of these known nucleic acids are contemplated for use with the invention. Typically, double-stranded DNA has a helical structure, but also can be found with other tertiary and quaternary structures. Single-stranded nucleic acids also can be found to exist in different structures such as hairpins or circular nucleic acids. Any of these forms and structures are contemplated for use with the invention. Nucleic acid tags preferably are DNA, more preferably are double-stranded DNA, and preferably are at least 10 base pairs long and up to about 1000 base pairs (bp). The nucleic acids can any suitable and convenient length from 10 to about 1000, for example 10 bp, 20 bp, 50 bp, 100 bp, 250 bp, 500 bp, 750 bp, or 1000 bp long.

Preferably, the nucleic acid tag is a DNA molecule with a molecular weight of less than about 650 kDa, more preferably less than about 200 kDa. Most preferred amplicons have a molecular weight below about 65 kDa, for example less than about 32 kDa or less than about 6 kDa. However, a sufficient number of base pairs are required for the sake of uniqueness and the minimum preferable number is 10 base pairs. Thus DNA nucleic acid tags are at least 10 base pairs long, preferably at least about 30 base pairs and most preferably about 50 base pairs. Using tags with a sufficient number of base pairs ensures that enough different sequences of the same length can be prepared to create a series of sequences with sufficient different permutations to create a unique set of tags. The actual sequence of the tag used in any application is not important, as long as the sequences are known or can be identified by specific amplification and analysis. The nucleic acid tags also should exhibit a well-defined and fairly narrow size distribution for easier and accurate amplification. This usually equates in terms of the chromatographic output to a size range under ±10 base pairs at ½ height around the average but can be a greater number.

Nucleic acid tags can be in the form of any suitable salt, including any alkali metal or alkali earth metal salt, or cations such as tetravalent ammonium, tetravalent phosphonium, and the like. The nucleic acids can be obtained from animal, plant, fungal or bacterial sources, or can be completely synthetic and non-naturally occurring. Random segments of nucleic acid from any source that have been re-ligated to form a new sequence also can be used. Nucleic acids obtained from a natural source can be any nucleic acid, including genomic DNA, nuclear DNA, mitochondrial DNA, chloroplast DNA, ribosomal DNA, transfer DNA, messenger RNA, and the like, of any sequence.

The inventive ink compositions contain the nucleic acid tags in a concentration of preferably less than about 0.1 weight percentage in the wet ink, for example about 0.1% by weight, or about 0.075% by weight or about 0.05% by weight. Even more preferably the polynucleotide concentration is less than about 1 part per million (ppm) in ink or less than 100 part per billion (ppb) in the ink, or less than 1 ppb in the ink.

D. Printing and Printed Marks

Printers which are capable of printing in production environments are suitable to use for the present invention to apply tagged marks to packaging, directly to an outer surface of items as part of the manufacturing process, directly to parts prior to assembly of the item so that the mark can be visible or hidden in the final product, or inside of items as they are manufactured or afterwards. These printers include inkjet types printing liquid ink (at printing temperature) or solid phase printers such as TTO (thermal transfer) printers that rely on heat to transfer ink from one media surface to another. Such printers are known in the art. The invention is suitable for use in continuous inkjet printers (industrial printers) for production line printing.

In order to carefully control the amount of ink printed and therefore the amount of the nucleic acid tag that is applied during printing, inkjet type deposition is preferred. Inkjet offers the general advantages of non-contact printing, high resolution, digital variable information, the ability to deliver relatively controlled doses of fluid, low consumable use, low VOC emissions, and ease of integration into manufacturing processes. Any suitable form of inkjet technology known in the art can be used. Ink jet printing can be broadly divided into drop-on-demand (DOD) printing and continuous inkjet (CIJ) printing. In drop-on-demand systems, ink droplets for printing are generated as and when required; in continuous ink jet printing, droplets are continuously produced under high pressure. CIJ inkjet printing is particularly preferred because it can print at relatively high standoff print distances from the nozzle (throw distance); it has very high production line speeds in comparison with DOD printing technologies, it employs solvent based inks exhibiting fast drying rates that enable the very fast production speeds, it is very easy to maintain, and it uses small, extremely easy to integrate printheads.

Inkjet printers are known in the art and are described in U.S. Pat. Nos. 9,044,954; 7,393,085; 8,789,923; and 8,960,886, which are hereby incorporated by reference in their entirety. CIJ printers most often operate by selectively charging and deflecting drops in flight to direct the ink to the proper location on the object to be printed. Drops are continuously generated at the nozzle by inducing break-off from a pressurized continuous stream of ink in the presence of a variable electrostatic field created by a charging electrode that places a discrete charge on selected drops. Drops subsequently pass through an electrostatic field wherein the field potential induces deflection on the charged drops in order to direct them to print or to direct them into an ink catcher to be reused in the ink system.

In binary array CIJ printing, the non-printed drops are charged and deflected to the gutter, and the printed drops are not charged. As is the case with DOD technology, the relative positions of the nozzles in the array in large part determine the relative position of the printed drops.

For DOD printing, a printhead device ejects ink droplets only when they are needed for imaging on the ink receiver, thereby avoiding the complexity of drop charging, deflection hardware, and ink collection. In one form of DOD, the ink droplet can be formed by means of a pressure wave created by the mechanical motion of a piezoelectric transducer behind or near the nozzle (the “piezo method”). In another mode of drop-on demand ink jet printing, the ink droplets are created by valves that open and shut independently thereby releasing ink that resides in a pressurized state behind the nozzle orifices (the “valve jet” method).

Thermal ink jet drop on demand (TIJ DOD) print heads produce ink droplets by thermal vaporization of the ink solvent. In the jetting process, a resistor behind the nozzle is heated rapidly to produce a vapor bubble which subsequently ejects a droplet from the orifice. This process is extremely efficient and reproducible. Modern TIJ print heads for industrial printing, like CU, are compact and capable of being integrated into different kinds of production lines so that they can operate under a range of environments. These print heads can print at a high resolution of 600 dots per inch in a given dimension or greater. Piezo DOD is similarly capable, but the price and physical footprint required for printing at such high resolutions with Piezo is far greater.

Although TIS printing systems have been available for over 30 years, nearly all of the commercial inks available for thermal ink jet systems have been water-based, i.e., they contain more than 50% water or other slow drying solvents such as propanol. Recently, volatile organic solvent compatible TIJ printheads have been developed such as those that employ HP45si cartridges or those used in the Videojet 8610® printing system. Thus, these printing systems are preferred for use with the inventive inks.

Printed marks are made from one or more droplets of ink can be in the range of about 100 fL to about 1 μL, depending on the printing technology. For example, TIJ ink drops are in the range of about 1 pL to about 100 pL, while CIJ ink drops are in the range of about 100 pL to about 10 nL. See also Table 1, below, for typical drop sizes in CIJ and TIJ printing. In general, the amount of ink to be deposited depends on the needs of the user and the concentration of nucleic acid tag in the ink. Currently, the most sensitive methods for DNA sequencing without amplification require at least about 1 ng of DNA per microliter of extract. Therefore, the methods of the invention preferably deposit less than 1 ng of DNA per coded article, so that ordinary detection methods not relying on amplification would not be possible. Thus, the marks according to the invention contain up to about 5 ng of extractable nucleic acid tag per microliter printed, and preferably less than about 1 μg of extractable nucleic acid tag and more preferably less than about 1 ng extractable nucleic acid tag. The final ink composition preferably contains 0.001 ppb to one part per thousand nucleic acid, more preferably 0.1 ppb to 100 ppm nucleic acid and most preferably 1 ppb to 1 ppm nucleic acid.

TABLE 1
Ink Delivery of DNA (ng).

| | | | | | | | | | | Exemplary Total DNA (ng) | | |
|---|---|---|---|---|---|---|---|---|---|---|---|---|
| | | | | | | Exemplary delivered ink (based on | | | | delivered per code (based | | |
| | | | | | | a typical printed code and typical | | | | on indicated wet ink DNA | | |
| | | | | | | ink properties and drop volumes) | | | | concentrations) | | |
| | Typical | | Typical ink properties | | | | total | wet | dry | 1 part | | |
| | drop | | solids | viscosity | density | pixel | volume | weight | weight | per | | |
| | volume | | (weight %) | (cP) | (g/mL) | count | (nL) | (mg) | (μg) | thousand | 1 ppm | 1 ppb |
| CIJ, large | 1.5 | nL | 25.0 | 5.0 | 0.86 | 336 | 504 | 0.43 | 108 | 433 | 0.43 | 0.00043 |
| nozzle | | | | | | | | | | | | |
| CIJ BX | 0.2 | nL | 16.0 | 4.0 | 0.85 | 336 | 67.2 | 0.06 | 9 | 57 | 0.06 | 0.00006 |
| array | | | | | | | | | | | | |
| printer | | | | | | | | | | | | |
| TIJ, | 10 | pL | 15.5 | 2.5 | 0.83 | 15,000 | 150 | 0.12 | 19 | 125 | 0.12 | 0.00012 |
| HP ®45 | | | | | | | | | | | | |
| TIJ, | 50 | pL | 15.5 | 1.5 | 0.83 | 15,000 | 750 | 0.62 | 96 | 623 | 0.62 | 0.00062 |
| Videojet ® | | | | | | | | | | | | |
| 8610 | | | | | | | | | | | | |

Systems suitable for the current invention in an embodiment are capable of tracking and internally validating the presence and nature of the oligonucleotide tag being used. For example, inkjet printers of the present invention can employ cartridges that contain a chip with non-volatile memory capability. Certain information may be recorded onto non-volatile ROM of the chip during production of the ink cartridge with the purpose of positively identifying either the ink, ink batch, tag identification, an encryption key code or even more detailed information such as tag composition or specifications that enable the tag to be decoded. Tag identity information can be manually programmed or automatically entered by a tag verification system using active analysis, for example.

U.S. Pat. No. 8,449,054 describes suitable CIJ inkjet systems that employ an ink cartridge containing an embedded data chip and is incorporated herein by reference. In general, these printers query the cartridge chip when it is inserted in order to confirm that the ink is of the proper variety per the software's instructions. Certain exemplary events can trigger the writing of information from the printer to the non-volatile memory on a cartridge including: usage of ink; expiry of ink; invalid use of cartridge in the wrong printer; removal and re-insertion of cartridge; etc. A log thus can be generated and maintained of how the ink cartridge has been used in practice, raise alarms for fraudulent use or even to invalidate the contents. The communication between the printer and the chip can occur via direct electrical contact or by near contact RFID. In addition, the communication for purpose of data exchange or tracking between the printer and the chipped cartridge may occur remotely, for example, by a GPS transponder or over a cellular network. Chipped ink cartridge configurations are compatible with any ink storage configuration including those used for binary array inks, TIJ ink cartridges, piezo ink cartridges, etc.

This type of system as described has many advantages, in various embodiments. End users benefit by being able to know they are using the right tag in the right system, which is especially useful if the end-user is printing more than one tag for different products. The printer software may be designed to furthermore reject the use of any other ink besides the one specified for a particular production line or a particular production job (i.e., comprising a particular message printing onto a particular kind of product) to reduce printing errors.

Because printing methods can be customized, the printing method for applying the tags of this invention can be the same as the inkjet coding method that end users already employ in their production process and no additional production process tools or steps are required. The additional burdens of tracking of ink or analysis of samples are relatively easy to implement by businesses using this approach. Tracking is relatively transparent and handled by software. The analysis expertise can be provided as a service by third parties with the capabilities to performing polynucleotide analyses.

Thus, according to certain embodiments of the invention, the polynucleotide tag manufacturers and users advantageously can track and reconcile the use of ink. For example, the batch information contained on the chip can be retrieved and a log describing which ink batches were used by which system can be generated by the printers' software. Ink level tracking is a standard feature of Videojet® 1000 Series' software, for example. In other embodiments, cartridge removal or insertion events are used to signal, respectively, the conclusion or initiation of ink consumption. The confluence of this information can be assembled into a database, for example, over a network for the purpose of tracking overall ink usage and help to proactively identify potential problems in the system level use or logistical distribution of ink.

In other embodiments, in addition to merely tracking the ink, a further verification step is possible where the system provides feedback that a print has occurred onto the article and this can be logged to a database record as a successful ‘print event’ of the tagged mark. The print event can be confirmed by either the print trigger relay that is normally used to signal when to print, or alternatively with a secondary optical system that analyses the printed article and confirms that the printed code is present, i.e., by successfully reading it. In a further, optional embodiment, this verification can be performed by an online verifier looking for a fluorescence trace, or the like.

In some embodiments of the invention, the mark need not be directly analyzed after printing or application to confirm that it does contain a tag. Instead, the printed code (overt or covert) contains a code, proprietary character, logo, trademark, or other detectable signature that indicates that the system has in fact successfully delivered the mark with the tag to the article. Other overt features can be such things as the code itself, a reference value contained in the code (normal or encrypted), an invisible luminescent material that emits light that is visible to an onlooker, and the like.

E. Nucleic Acid Recovery and Analysis

DNA in printed marks can be recovered and sequenced by any suitable means known in the art, including physical sampling or remote querying. Physical sampling of marks generally is conducted using any convenient approach, including swabbing, dissolving, scraping, cutting, or abrading to remove a sample of the mark or the entire mark. Swabbing can be conducted using a gentle or mild solvent such as water, saline solution, or a commercially available DNA extraction solution, or any convenient buffered aqueous solution containing aids to DNA dissolution, preservation or purification, such as a detergent, a DNA denaturant, a proteinase, an RNAse, a chelating agent, a solvent, and the like. Alternatively, an organic solvent appropriate for dissolving the dried ink mark can be used. In some cases, water is preferred, for example, because it is non-destructive and the printer mark containing information remains intact after analysis. In some cases, complete removal of the mark may be necessary.

The ink or removed tags can be analyzed using any one of the many known and available polynucleotide and oligonucleotide analytical methods. Testing can be performed merely for the purpose of confirming the presence of the known nucleic acid sequence, or complete forensic level analysis and/or a complete characterization of the nucleic acid can be performed. Any suitable analysis method for nucleic acid maybe be used. For example, the following methods can be used for analysis of the marks and their extracted contents: qualitative analysis such as probing the sample with a labeled complementary probe and detecting binding (e.g., using fluorophore-labeled probes followed by an assay to detect bound fluorescence), or quantitative analysis such as PCR amplification/separation followed by nucleotide sequencing. Using current methods, these types of qualitative and quantitative methods can be used by the end user or can be sent to an outside laboratory for the analytical work. Simple hybridization probe methods take advantage of the ability of a labeled single-stranded nucleic acid sequence to specifically hybridize to a complementary sequence to be detected. Therefore, such probes have a sufficient length to ensure specificity and high affinity and avidity in an assay. Hybridization probes generally should be long enough to avoid hybridizing with similar sequences (potentially creating a false positive) but not so long as to decrease efficiency of binding. In general a probe of this type is about 20-200 nucleotides long or can be up to about 1000 nucleotides, depending on the hybridization conditions and the sample being assayed. This can easily be determined by the person of skill in the art, and will depend on factors such as the length of nucleic acid to be detected, its purity and concentration, the assay volume and type, and physical parameters such as temperature, pH, and osmolality which can effect hybridization.

Illustrative sequencing methods that can be used to identify and sequence DNA include but are not limited to chemical cleavage sequencing, chain terminator methods, sequencing by hybridization, sequencing by synthesis, primer extension using synthetic location-specific primers (Sanger methods), wandering spot analysis, DNA polymerase catalysis, specific nucleotide labeling, stepwise base-by-base analysis, and also high-throughput methods such as automated “massively parallel” sequencing, phred quality score sequencing, massively parallel signature sequencing, and the like. RNA sequencing generally is performed by creating a complementary DNA molecule from the RNA and sequencing that DNA.

PCR screening of any type known in the art is contemplated for use with the invention. This can include any form of PCR methods compatible with screening, including qualitative, real time, quantitative, nested, touchdown, fast, direct, multiplex, lab on a chip, and the like. PCR also can be combined with any other method for the purpose of separating or purifying the amplified product including, for example, gel electrophoresis, capillary electrophoresis, microfluidic separation, etc., and any DNA separation or analytical methods known in the art. The PCR methods preferably are optimized with respect to the nucleic acid (or template DNA) for best specificity, efficiency and fidelity for identification. Potentially controlled conditions include those such as denaturant type (if required), buffer conditions, annealing step adjustments, temperature, cycle times, primer composition, free base pair composition, extension step adjustments, polymerase type, etc. Primers can be any length, though it is common to have primers of at least 15 bases in length for adequate specificity, and preferably such primers are about 15 bases to about 50 bases, or about 18 bases to about 30 bases, or about 15 bases to about 25 bases, and most preferably about 16 to about 22 bases in length.

A printed mark that contains under 1 part per thousand of a particular nucleic acid (far more than what is required for the methods to function) also can contain other naturally occurring strands, but a user attempting to reverse engineer and determine the sequence of the tag (without knowing the sequence), will encounter great difficulty. Using PCR methods, specifically identifying a unique DNA contained within the ink realistically can be accomplished only if a specific procedure and set of raw materials are used. Thus, the approach is highly secure since the nucleic acid tag cannot be reproduced unless the base sequence of the DNA is revealed or isolation material and methods are revealed. They are not present in high enough concentration in the marks to be vulnerable to most general sequencing methods.

However, knowing the exact length and sequence of the tag DNA in the mark allows the user to design, prepare and use a specific primer or primer set with an adequately high number of nucleotides so that the complementarity to the nucleic acid is highly specific for the particular tag in question. In other embodiments, the length and sequence do not need to be known if primers for amplification of the DNA are known or available. This, combined with the statistically improbable likelihood that a similar DNA sequence will be present in any quantity in the tag that would be amplified by the primers or that would hybridize with the specific probe provide near 100% assurance when a user is seeking to confirm the identity of a known tag in a mark.

F. Applications

The embodiments of this invention typically are used when a user requires more than average security to confirm the authenticity of a product, and when serialization alone is not sufficient or not possible. However, these inks and methods can be used to tag any item, particularly items of commerce.

In some embodiments, the nucleic acid tagged ink is printed directly onto electronic assemblies or sub-components. Often electronic components can be very small, or have small effective printable areas. Hence, a suitable mark for a component might be a series of small characters, a barcode, a logo, or some series of printed dots of a desired pattern. The mark in this embodiment needs to be reliably delivered within an area that may be on the order of hundreds of microns in diameter.

In some embodiments, tagged marks are used in the pharmaceutical industry to print authentication marks onto pharmaceutical products such as tablets, capsules, caplets, and the like or onto their packaging such as prefilled syringes, bottles, vials, bags, blister packs, boxes, cartons, or any container, in order to reduce the substantial health risks or liability that can arise from mis-labelling or counterfeit goods in this industry. Particularly with direct pill printing, there is limited surface area to print a mark and it is of advantage to print with a non-contact method to protect what may be a fragile surface. Ideally, the tag therefore is printed along with the routine serialization information that is required as part of pharmaceutical packaging to protect consumers from black market or diverted drugs.

In some embodiments, tagged marks are used by the cosmetics industry to protect their brand and keep counterfeiters from selling what would appear to be identical products.

In some embodiments, tagged marks are used for the purpose of preventing grey market redistribution heavily regulated and/or taxed items such as alcohol or tobacco products which are typically taxed heavily at the point of sale. In this embodiment, preferably the security features of the printed mark (e.g., serialization) can be combined with the use of a nucleic acid tag to provide a different means to authenticate an item.

In summary, the invention can be used to tag any product, individual component of a product, packaging, or label to provide secure traceable authentication of goods in any industry. Any product that is high-value or luxury, heavily regulated or taxed, frequently counterfeited, or that would benefit from a method of authentication preferably will take advantage of this method of marking. Such industries can include but are not limited to electronics, pharmaceuticals, cosmetics, fashion, jewellery, art, distilled spirits and wine, tobacco, investment products, currency, and the like, but can include any person or organization that requires some kind of security element on their goods that is directly verifiable with high assurance and that does not necessarily rely on databases or secondary tracking measures.

Additional advantages of this system of marking include:

1. Polynucleotide tags are stable during storage and inkjet compatible as opposed to dense pigments or unstable conventional security tags. Polynucleotides are also stable in the environmental conditions to which the printed marks will be exposed.
2. The mark can be applied with inkjet (or TTO) directly onto goods, primary containers or packages at the desired location in the manufacturing process with good drying, adhesion, good code readability (or relative covertness when required).
3. The tag can be applied at the same time that the variable information inkjet code is normally provided, saving production steps and the information contained in the code can be used to signal DNA content or augment the security of the DNA.
4. Information contained on a chipped printer cartridge can be used to validate a DNA tag, provide assurance to customers, or to track the DNA tag.
5. The printer or secondary vision systems can be used to confirm that the goods have been properly marked.
6. The invention further includes the aspects combining the serialization of products using printed codes which is established in the art, with the benefits of a chemically detectable tag. The tags can be reliably validated in the printer using a handshake between the printer software and data chips that reside on the ink cartridges which helps guarantee ink delivery and track ink use and distribution.

G. Results

The inkjet inks of the present invention have been found to show good nucleic acid compatibility, which allows the nucleic acid to remain dissolved and/or dispersed within the ink for a practical usage life without detriment to system reliability, when tested in a CIJ printer which typically subjects inks to very harsh use conditions. Unlike other kinds of security tags based on luminescent pigments, nucleic acids have not shown any tendency for rapid precipitation in the ink within the printer. They have not tended to aggregate or clog absolute rated membrane filters housed within the CIJ printer's ink system. Further, they are not hard or abrasive materials and thus do not have a tendency to damage the system in use.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Unless defined, otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

### Example 1. Printing with Methanol and Methyl Ethyl Ketone Based Inks

Inks of formulations A, B, and C, having the components shown in Table 2, were made using standard inkjet practices as known in the art. Then, roughly 1 mL of solution containing a nucleic acid polynucleotide was added directly to approximately 750 mL or each of the inks. The concentration of polynucleotide used was less than 0.1% by weight. See Table 2.

TABLE 2
Exemplary Ink Compositions (not including
polynucleotide solution).

| | Weight Percent | | |
|---|---|---|---|
| | Formulation | Formulation | Formulation |
| Material Description | A | B | C |
| MEK | 42.0 | | 34.7 |
| Methanol | | 63.8 | 29.0 |
| Benzyl alcohol | | 3.2 | 5.2 |
| Glycol ether PM | | | |
| Glycol ether DE | | 1.6 | |
| Denatured Ethanol | 11.0 | | |
| Water | 0.1 | 2.1 | 0.1 |
| Varcum ® 29108 | | | 17.1 |
| Scholle ® 6558 | 22.5 | 5.3 | |
| Xiameter ® RSN-02323 flake | 2.0 | | |
| resin | | | |
| Joncryl ® 611 | | | 9.4 |
| Joncryl ® 678 | | 15.8 | |
| CAP 482-0.5 | 3.2 | | |
| Plasticizer 8 | 3.0 | 0.5 | |
| 2-Pyrollidone | 3.0 | 2.1 | |
| Uvitex ® OB | 0.2 | | |
| Tetrabutylammonium | 1.0 | | |
| hexafluorophosphate | | | |
| Lithium nitrate | | 0.3 | 0.4 |
| Isopropyl acetate | 12.0 | | |
| Keyazine crystal Violet 6B | | 1.4 | |
| Solvent Orange 25 | | 3.8 | |
| Solvent Black 7 | | | 3.1 |
| Silwet ® L7622 | | | 0.1 |
| Silicone fluid 69 | | 0.1 | |
| BYK - 065 | | | 0.9 |
| TOTAL | 100.0 | 100.0 | 100.0 |
| Percentage of solvent | 73.2 | 72.6 | 69.9 |

The solvents shown in the table are generally greater than 98% pure and are available from a wide range of commercial sources. Denatured ethanol is Duplicating fluid #5 available from Nexeo® Solutions. Varcum 29108 is a reactive phenolic resin available from Sumitomo Chemical®, Inc. Scholle 6558 is a 16% solution of IPA wetted (ca. 6% IPA content) nitrocellulose in MEK. available from Scholle®, Inc. Xiameter RSN-0233 Flake resin is a siloxane resin available from Dow Corning®, Inc. Joncryl 611 and Joncryl 678 are solvent soluble styrene acrylic resins available from BASF®, Inc. CAP 482-0.5 is a cellulose acetate ester resin available from Eastman Chemical®, Inc. Uvitex OB is an invisible, UV excitable fluorescence dye with a blue emission available from Ciba-Geigy®, Inc. Tetrabutylammonium hexaflurophosphate and lithium nitrate are salts available from Sigma Aldrich®, Inc. Keyazine Crystal Violet 6B is a triarylmethane violet dye available from Milliken Chemical®, Inc. Solvent Orange 25 is an azo based orange dye available from BIMA 83®, Inc. Solvent Black 7 is available from Orient USA®, Inc. Silwet L7622 and Silicone fluid 69 are surfactants available from Momentive Performance Materials®, Inc. BYK-065 is a defoamer available from BYK Chemie®, Inc.

Each of the formulations in Table 1, with the addition of polynucleotide, were printed using a Videojet® 1000 Series' continuous inkjet printer. Formulations A and B were each printed onto substrates including PET plastic, aluminium, glass and coated paper. Formulation A produced a nearly invisible code when printed and provided very good adhesion to the above substrates. It also was fluorescent under UV light illumination. Formulation B provided a black code and very good adhesion to aluminium, glass and coated paper. Both formulations A and B dried on the substrates within about 3 seconds after printing. For both examples on PET and aluminium, the codes did not change in their appearance after hard rubbing with a human thumb ten times. The codes also did not smear or show any visibly reduced contrast by rubbing with a water-wetted thumb for 10 seconds. Formulation C also provided a black code and was cured on an aluminium sample by placing in an oven and heating (150° C. for 30 minutes). After curing, the code could not be removed by wiping ten times with an acetone soaked swab. See Table 3 for a summary of results including confirmation of presence of the polynucleotide tag by PCR type analysis.

TABLE 3
Printing and Analysis Results.

| | Poly- | | | Poly- |
|---|---|---|---|---|
| | nucleotide | Poly- | | nucleotide |
| | concentra- | nucleotide | | confirmed in |
| Material | tion, weight | confirmed | Ink code | dry ink from |
| Description | percentage | in ink | printed | printed codes |
| Formulation A | <0.1 | Yes | Yes, invisible | Yes |
| Formulation B | <0.1 | Yes | Yes, visible | Yes |
| Formulation C | <0.1 | Yes | Yes, visible | Not tested |

### Example 2. Stability and Detectability of Nucleic Acid after Printing Operation

Formulations A and B (including polynucleotide tag; see Table 2) were subjected to simulated aging for a period of 1 year at high temperature (50° C. or 60° C.) to determine the stability of DNA upon storage in the bottle. After simulated aging, the ink was subjected to filtration tests and standard properties tests (viscosity, pH, conductivity). No property had changed more than 10% from initial. Filtration was still possible to a 1 micron absolute level.

Ink Formulation A was also tested in a Videojet® printer for a period in excess of 940 hours of continuous recirculation without replenishing ink within the system. Periodic print quality checks were conducted. The 940 hours included a period of approximately 600 hours at room temperature and a further approximately 340 hours at 45° C. and 80% relative humidity ambient conditions. PCR analysis of the printed codes acquired after this period confirmed unequivocally the presence of the DNA tag. The results confirm that the DNA tag was not significantly degraded by the high temperature, high shear, and oxidative conditions typical within a CIJ printer. See Table 4.

TABLE 4
Stability Testing.

| | | | Polynucleotide |
|---|---|---|---|
| | | | confirmed in |
| | Stability of | Polynucleotide | printed codes |
| | ink after 1 year | confirmed after 1 | after testing |
| Material | of simulated | year of simulated | in printer for |
| Description | shelf life | shelf life | 944 hours |
| Formulation A | Stable | Yes | Yes |
| Formulation B | Stable | Yes | Not tested |

### REFERENCES

All references listed below and throughout the specification are hereby incorporated by reference in their entirety.
1. United States Patent Publication No. 2018-0016627
2. United States Patent Publication No. 2017-0021611
3. United States Patent Publication No. 2015-0107475
4. United States Patent Publication No. 2009-0311415
5. United States Patent Publication No. 2008-0190808
6. United States Patent Publication No. 2015-0240297
7. United States Patent Publication No. 2010-0285985
8. United States Patent Publication No. 2005-0112610
9. United States Patent Publication No. 2007-0048761
10. United States Patent Publication No. 2014-0106357
11. United States Patent Publication No. 2014-0272097
12. United States Patent Publication No. 2014-0102899
13. United States Patent Publication No. 2016-0076088
14. United States Patent Publication No. 2016-0362723
15. United States Patent Publication No. 2003-0186257
16. United States Patent Publication No. 2012-0162701
17. United States Patent Publication No. 2008-0169926
18. United States Patent Publication No. 2017-0158897
19. U.S. Pat. No. 9,266,370
20. U.S. Pat. No. 8,114,207.
21. U.S. Pat. No. 9,919,512
22. U.S. Pat. No. 9,171,443
23. U.S. Pat. No. 5,139,812
24. U.S. Pat. No. 5,885,678
25. U.S. Pat. No. 6,536,672
26. U.S. Pat. No. 7,710,269
27. U.S. Pat. No. 9,323,485
28. U.S. Pat. No. 4,892,775
29. U.S. Pat. No. 8,449,054
30. International Patent Application No. WO/2014/164958A1
31. International Patent Application No. WO/2007/015261A3
32. International Patent Application No. WO/2014/0119099A1
33. International Patent Application No. WO/2013/170009A1
34. International Patent Application No. WO/2014/059061A1
35. International Patent Application No. WO/2014/062754A1
36. International Patent Application No. WO/2004/094713A2
37. International Patent Application No. WO/2004/094713A3
38. International Patent Application No. WO/2006/127558A2
39. International Patent Application No. WO/2006/127558A3
40. International Patent Application No. WO/2000/070537A1
41. International Patent Application No. WO/2006/092286A1
42. International Patent Application No. WO/2001/062860A1
43. European Patent No. EP1856277B1
44. European Patent No. EP2894228A1
45. European Patent No. EP1246945B1
46. European Patent No. EP2909822A1
47. European Patent No. EP2909822A4
48. European Patent No. EP2969586A1
49. European Patent No. EP1384790A2
50. European Patent No. EP1384790A3
51. Japanese Patent Application No. JP2002340894A
52. Japanese Patent Application No. JP2003525429A
53. Japanese Patent Application No. JP2004331832A
54. Japanese Patent Application No. JP2013000047A
55. Japanese Patent No. JP6154441B2

1. A tagged inkjet ink composition, comprising:
(a) about 35% to about 95% of an organic solvent; and
(b) an amplifiable nucleic acid tag.

2. The tagged inkjet ink composition of claim 1, wherein the solvent is selected from one or more of the group consisting of a ketone, an alcohol, an ester, and an ether.

3. The tagged inkjet ink composition of claim 1, wherein the solvent is methanol, ethanol, methyl ethyl ketone, acetone, or a mixture thereof.

4. The tagged inkjet ink composition of claim 1, which comprises less than 5% added water.

5. The tagged inkjet ink composition of claim 1, wherein the nucleic acid is double-stranded DNA or a salt thereof.

6. The tagged inkjet ink composition of claim 5, wherein the salt is an alkaline or alkaline earth salt.

7. The tagged ink composition of claim 1, wherein the nucleic acid has a molecular weight less than or equal to 650 kDa.

8. The tagged inkjet ink composition of claim 1, wherein the nucleic acid is at least 10 base pairs long.

9. The tagged inkjet ink composition of claim 1, wherein the nucleic acid is at least 50 base pairs long.

10. The tagged inkjet ink composition of claim 1, wherein the nucleic acid concentration in the ink composition is less than or equal to 0.1% by weight.

11. The tagged inkjet ink composition of claim 1, wherein the nucleic acid concentration in the ink composition is less than or equal to 1 part per million.

12. The tagged inkjet ink composition of claim 1, wherein the nucleic acid has a molecular weight of less than 100 kDa and the nucleic acid concentration in the ink composition is less than 0.1% by weight.

13. The tagged inkjet ink composition of claim 1 which is stable and the nucleic acid is detectable in a printed mark after bottle storage for at least 1 year and during operation within a continuous inkjet printer for more than 900 hours.

14. The tagged inkjet ink composition of claim 1, further comprising a resin.

15. The tagged inkjet ink composition of claim 1, wherein the resin is cross-linkable or curable.

16. The tagged inkjet ink composition of claim 14, wherein the resin is a styrene acrylic resin.

17. The tagged inkjet ink composition of claim 14, wherein the resin is a modified cellulose resin.

18. The tagged inkjet ink composition of claim 1, further comprising a colorant.

19. The tagged inkjet ink composition of claim 18, wherein the colorant is visible.

20. The tagged inkjet ink composition of claim 18, wherein the colorant is a luminescent compound which can be visible or invisible to the naked eye.

21. The tagged inkjet ink composition of claim 1, wherein the printed mark dries in less than 3 seconds and exhibits smear resistance to aqueous fluids.

22. A method of tagging an article, comprising applying the tagged inkjet ink composition of claim 1 to the article or packaging for the article to produce a mark.

23. The method of claim 22, wherein the tagged inkjet ink composition is applied by continuous inkjet printing to produce a mark.

24. The method of claim 22, further comprising analyzing the mark to determine the presence of the nucleic acid tag.

25. A system for applying a tagged ink composition to produce a mark on an article, comprising:
(a) an inkjet printer;
(b) an ink cartridge containing a nucleic acid tagged inkjet ink composition according to claim 1 and incorporating a data chip that contains actual information, relational information or both regarding the specific nucleic acid tag contained in the ink,
wherein the data chip, via direct contact between the inkjet printer and the data chip, provides confirmation of the identity of the nucleic acid tag present in the ink being printed.
